(19) United States
(12) Patent Application Publication
(10) Pub. No.: US 2023/0277114 A1
(43) Pub. Date: Sep. 7, 2023

(54) INVESTIGATION AND REMOTE-THERAPY SYSTEM FOR MITIGATING NEURAL-DISORDER SYMPTOMS

(71) Applicant: Adam Benjamin Tannenbaum, Ashdod (IL)

(72) Inventor: Adam Benjamin Tannenbaum, Ashdod (IL)

(21) Appl. No.: 17/687,651

(22) Filed: Mar. 6, 2022

(51) Int. Cl. A61B 5/377; A61B 5/00

(52) U.S. Cl. CPC A61B 5/377; A61B 5/0006; A61B 5/0008; A61B 5/0013; A61B 5/6802; A61B 5/6813

(57) ABSTRACT

The invention herein disclosed is a dual-function system that is used to investigate the response by an individual with neural-disorder symptoms to a variety of stimuli having different intensities and durations. Once a combination of stimuli and their settings is found to produce minimal frequency and severity of neural-disorder symptoms, the invention is then used to apply those stimuli and settings that minimize frequency and severity of symptoms as a therapeutic tool.

### TECHNICAL FIELD

The invention is a system used to investigate the effect of applying a variety of stimuli to an individual while measuring the frequency and severity of neural-disorder symptoms.

### BACKGROUND OF INVENTION

People who have such neural disorders as stammering (e.g. stuttering) or facial tics, when exposed to various stimuli such as heat, cold, vibration, electrical shock will exhibit varying degrees of frequency and severity of those disorders.

By applying various stimuli at specific intensities and durations, it has been found that each individual will respond to some combination so as to exhibit a significant decrease in frequency and severity of a particular neural-disorder symptom.

By carefully investigating the effect of different stimuli on an individual and his/her neural-disorder symptom frequency and severity, one can find a combination of stimuli, with specific, fixed intensity and duration, that will significantly mitigate the frequency and severity of a neural-disorder symptom.

Once an individual's response to various stimuli at specific intensities and durations has been investigated, it is possible to find a set of stimuli at specific intensities and durations that minimize the frequency and severity of a neural-disorder symptoms.

By using the stimuli and settings that produce minimized frequency and severity of a neural-disorder symptom, one can then make use of that set of stimuli and its settings as a means of therapy aimed at reducing the frequency and severity of such neural-disorder symptoms.

### BRIEF DESCRIPTION OF INVENTION

The invention is a system that enables a user to investigate various stimuli at various settings of intensity and duration while monitoring pulse rate, blood pressure, and brain activity to ensure that any adverse reaction is quickly noted, and by observing the frequency and severity of neural-disorder symptoms and resistance to body motion. In this way, the use of stimuli and body motion to mitigate neural-disorder symptoms can be investigated while ensuring that patient safety is carefully monitored.

Once a set of stimuli and specific intensities and durations are correlated with significant neural-disorder symptom mitigation, that stimuli and its settings may be used as a therapeutic aid for reducing neural-disorder symptom frequency and severity overall.

The system comprises an electronic control subsystem interfaced with transducers that can provide such stimuli as heat, cold, vibration, and/or mild electrical shock applied directly to an individual while concurrently monitoring blood pressure, pulse rate, body-motion resistance and brain activity.

Once an optimal set of stimuli and their settings has been found, the settings can be programmed into the electronic controller to apply those specific stimuli, intensities and durations as a therapeutic means of mitigating neural-disorder symptoms.

### BRIEF DESCRIPTION OF DRAWINGS

FIG. 1 shows an embodiment of the invention system

FIG. 2 depicts the functional components of the control subsystem

FIG. 3 is a flow diagram of an investigation method using the system

FIG. 4 is a flow diagram of a therapy method using the system.

### DETAILED DESCRIPTION OF INVENTION

Many people suffer with such neural disorders as stammering (or stuttering) and facial tics. The symptoms of such disorders can be debilitating.

It was found that people suffering from such symptoms would exhibit lower frequency and severity during therapy sessions where stimuli, such as coldness, heat, vibration and mild electrical shock was applied to them for specific intensity and duration. In addition, it was found that resistance to requested body motions could also be correlated to symptom mitigation.

Using a cobbled-together system of stimuli generators, investigators found that individuals would benefit from specific stimuli set at specific intensities for specific durations, but that there was significant differences in those settings among individuals. Thus, it was necessary, first, to investigate how different stimuli applied for different durations at different intensities affected the frequency and severity of neural-disorder symptoms.

Once an individual's response to various stimuli and intensity/duration settings was explored, and a combination was found to markedly reduce frequency and severity of symptoms, those stimuli and settings could then be used for therapeutic purposes on that individual.

The invention herein disclosed is a system comprising electronic control of a variety of stimulation transducers combined with constant monitoring of an individual's blood pressure, pulse rate, brain activity, and resistance to body motion. In essence, it creates a closed-loop system wherein various stimuli at different intensities and durations are explored while monitoring an individual for frequency/severity of neural-disorder symptoms, and monitoring an individual for adverse changes in vital indications.

The same system can be used for essentially two purposes: investigating application of stimuli to an individual while capturing changes in neural-disorder symptom frequency and severity; and for applying specific stimuli at specific settings of intensity and duration, therapeutically, based upon finding combinations that produce marked decrease in symptom frequency and severity.

The benefits in having both the investigation and therapy applications enabled by the same system are consistency and speed of set up and use, and ensuring that there is consistency in the accuracy of settings in the transition from investigation to therapy.

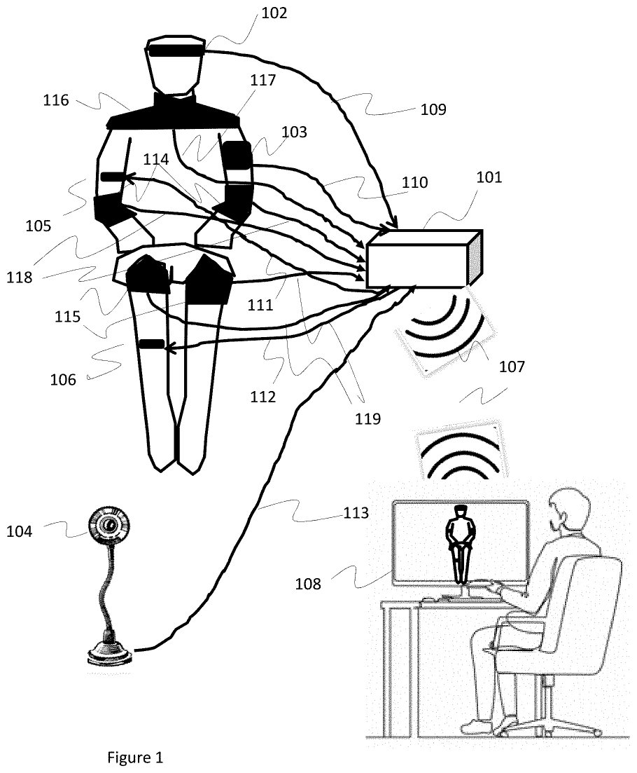

FIG. 1 illustrates an exemplary embodiment of the system comprising a control subsystem (101), a variety of stimulus transducers (105 and 106), a variety of vitals sensors (102 and 103), body-joint-cuff sensors (114 and 115) a camera/microphone peripheral (104), a practitioner subsystem (108), and wireless interface (107) between control subsystem and practitioner subsystem. The transducers, sensors, camera, body-joint-cuff sensors and microphone are interfaced to the control system via conductive paths (110, 111, 112, 113, 116, 118 and 119). Note that body-joint-cuff sensors may also be worn on ankles, wrists and knuckles (not shown).

The same transducer can be used to convert a signal from the control system to application of cold or warm stimulus. For example, an invention from University of California San Diego is a device that is worn like a wrist band that is reversible in terms of inner and outer layer. When worn with one side touching the skin, control signals can produce a safe feeling of coldness (e.g. a nominal temperature of 45 degrees F.), and when reversed, control signals can produce a safe feeling of heating (e.g. a nominal temperature of 105 degrees F.). Similarly, there are transducers that can apply safe levels of vibration and mild electrical shock (e.g. electro-stimulation) based on control signals.

Electrodes that are attached to the scalp (102) can provide electroencephalography (EEG) signals showing changes in brain activity; and a cuff (103) worn on an arm can, under signal control, provide pulse and blood pressure signals. The camera/microphone/wireless headset peripheral (e.g. a webcam and wireless headset subsystem) provides visual and audible feedback as investigation or therapy methods are invoked. The body-joint-cuffs (114 and 115) are worn over the elbows and knees and can detect the dynamics of arm folding and knee flexing so as to provide objective measures of resistance to such body motion.

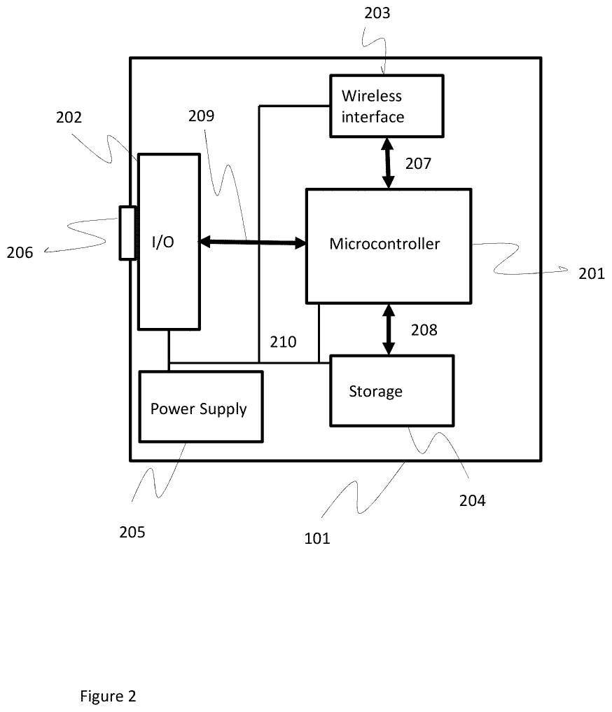

FIG. 2 is an exemplary embodiment of the control subsystem (101) comprising a microcontroller (101) interfaced to an input-output subsystem (202), a wireless interface subsystem (203), and a data storage subsystem (204). The input-output subsystem interfaces with a set of external connectors (206) to which transducers, sensors, and webcam can be connected. The control system and its subsystems are powered by a power supply (205). Bi-directional buses (207, 208 and 209) provide a path for conveying signals to and from the microcontroller and the other subsystems. And a common power bus (210) conveys power from the power supply to all subsystems comprising the control subsystem.

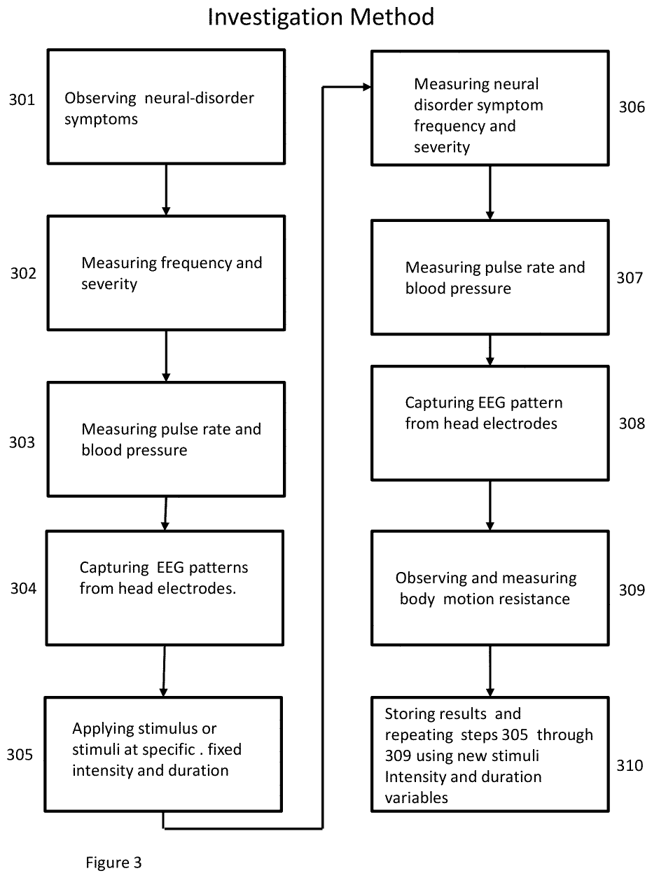

FIG. 3 shows an exemplary embodiment of an investigation method using the system of FIG. 1 wherein an individual is observed exhibiting neural-disorder symptoms (301), then measuring frequency and severity (302) while concurrently monitoring blood pressure and pulse rate (303), while capturing brain activity (304), applying at least one stimulus at a specific setting (305), then measuring disorder symptom frequency and severity (306), measuring blood pressure and pulse rate (307), capturing brain activity (308), observing and measuring body motion resistance (309) then storing results for all as stimuli settings are changed (310). In essence, the investigation method comprises applying different sets of stimulus intensity and duration while measuring effects upon neural-disorder symptoms and vitals. Later, the results can be scanned to find stimuli and settings that produce significantly reduced symptom frequency and severity.

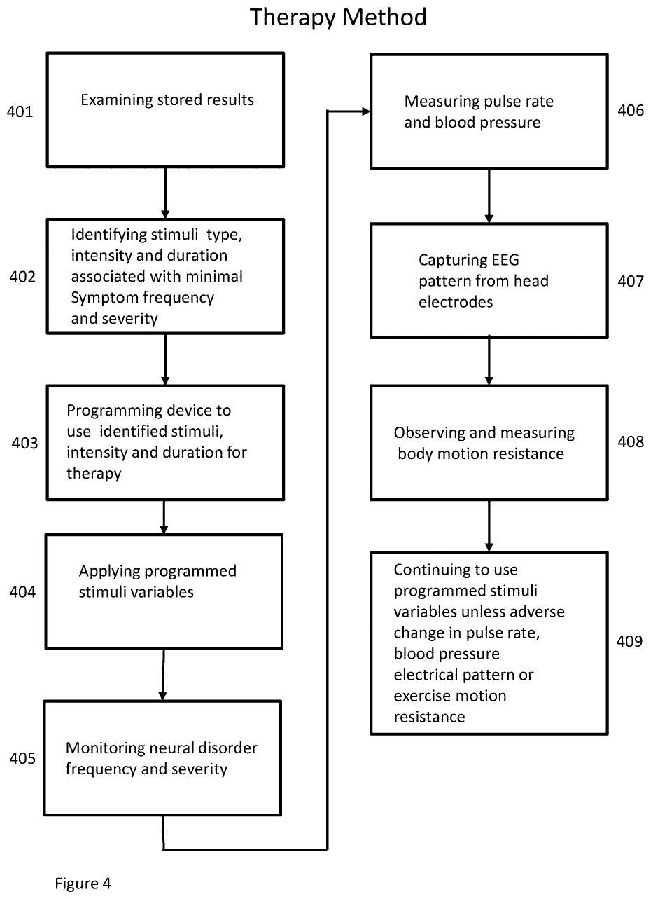

After the investigation has been completed, the therapy method embodiment shown in FIG. 4 may be applied. Here, the results of investigation are examined (401), stimuli and settings are identified that produce minimal symptom frequency and severity (402). Then the device is programmed with the identified stimuli and settings (403) which are then applied to the individual (404) while monitoring symptom frequency/severity (405), measuring vitals (406), capturing brain activity (407), observing and measuring resistance to body motion (408) and continuing to use these settings, therapeutically, unless adverse changes to vitals, brain activity, or resistance occur (409). In the case where an individual's response to therapeutic stimuli and settings changes adversely, a new investigation can be done to find a new stimuli/setting combination.

It should be noted that the invention is intended to be used so as to avoid a practitioner making physical contact with an individual during either investigation or therapy. The practitioner may be in the same room or remotely located. Where the practitioner is remotely located, the wireless interface can convey signals to and from the practitioner via the Internet using WiFi and virtual-private-network (VPN) link to ensure HIPAA privacy regulations are preserved. Vital sensors and body-joint-cuff sensors may be separate components or integrated into a garment or garments. The drawing shows discrete conductive paths for each sensor and transducer but an integrated subsystem can be added wherein the interface to the control subsystem comprises fewer conductive paths using a digitized serial data stream.

It should be noted that the disclosure and drawings comprise stimuli of heat, cold, vibration and mild electrical shock but other stimuli, such as visual images and colors, or sounds, may be found to have therapeutic value. In such case, the same system can be used to apply visual or auditory stimuli using an appropriate transducer. Thus, the drawings and exemplary descriptions should not be seen as limiting the invention scope or content.

What is claimed is:

1. A system comprising:
a control subsystem;
said control subsystem comprising:
a microcontroller subsystem operative to receive sensor inputs and based on program algorithms to convey control signals to at least one transducer;
a wireless-interface subsystem operative to receive and transmit wireless signals, to convert received wireless signals into digital data; and to convert input data signals into wireless signals;
an input-output subsystem operative to convey sensor signals to said microcontroller subsystem and to convey control signals from said microcontroller subsystem to said at least one transducer;
a data storage subsystem operative to receive and store data from said microcontroller subsystem and provide stored data to said microcontroller subsystem;
a power-supply subsystem operative to supply operating power to said microcontroller, wireless-interface, input-output and data-storage subsystems;
a camera, microphone, and wireless headset peripheral operative to capture images and audible sounds and convert said images and said sounds into electrical signals for conveyance to said control subsystem, and to convert verbal-instruction data into audible sound output;
a first transducer operative to convert electrical signals into thermal energy at a nominal temperature of 105 degrees F. and to convert electrical signals into thermal energy at a nominal temperature of 45 degrees F.;
said first transducer is operative to make contact with skin and convey a stimulus evoking a sensation of heat;
said first transducer is operative to make contact with skin and convey a stimulus evoking a sensation of cold;
a second transducer operative to convert electrical signals into physical vibration;
said second transducer is operative to make contact with skin and convey a stimulus evoking a physical buzz sensation;
a third transducer operative to convert electrical signals into electric impulses;
said third transducer operative to make contact with skin and convey a stimulus evoking a sensation of electrical shock;
a headband sensor comprising electrodes operative to detect brain activity;
said headband sensor is operative to convey electroencephalography EEG signals to said control subsystem;
a sensor cuff operative to detect pulse rate and blood pressure;
said sensor cuff operative to convey electrical signals to said control subsystem;
a body-joint-cuff sensor operative to detect resistance to joint flexure and to convey said electrical signals to said control subsystem;
a practitioner subsystem operative to receive wireless signals conveyed by said control subsystem;
said practitioner subsystem operative to display images and reproduce sounds conveyed by said camera and microphone peripheral to said control subsystem, and conveyed by said control subsystem to said practitioner subsystem; and
said practitioner subsystem operative to respond to practitioner keyboard inputs, produce practitioner-control electrical signals therefrom, and convey via wireless signals said practitioner-control electrical signals to said control subsystem.

2. A method for investigating the effects of stimuli on neural-disorder symptoms using a system as claimed in claim 1 comprising:
a. noting neural-disorder symptoms;
b. measuring frequency and severity of said neural-disorder symptoms;
c. measuring pulse rate and blood pressure;
d. capturing EEG signals from head electrodes;
e. applying at least one stimulus having fixed intensity and duration;
g. measuring, again, said neural-disorder symptom frequency and severity;
h. measuring said pulse rate and said blood pressure;
i. capturing said EEG signals from said head electrodes;
j. observing and measuring body motion resistance; and
k. storing results and repeating steps e through j with new, changed, fixed intensity and duration.

3. A method for providing therapy to mitigate neural-disorder symptoms using a system as claimed in claim 1 comprising:
a. examining stored investigation method results;
b. identifying stimuli type, intensity and duration associated with minimal neural-disorder symptom frequency and severity;
c. programming a system as in claim 1 with settings identified in step b;
d. applying programmed stimuli variables to individual;
e. monitoring neural-disorder symptom frequency and severity;
f. measuring pulse rate and blood pressure;
g. capturing said EEG signals from said head electrodes;
h. observing and measuring body motion resistance; and
i. continuing to apply said programmed stimuli variables while monitoring vitals for adverse reactions.
